(19) United States
(12) Patent Application Publication
(10) Pub. No.: US 2011/0123502 A1
(43) Pub. Date: May 26, 2011

(54) METHOD FOR OBTAINING TREG-CELLS

(76) Inventors: Simon C. Barry, South Australia (AU); Richard James D'Andrea, South Australia (AU); Jonathon F. Hutton, South Australia (AU)

(21) Appl. No.: 12/528,292

(22) PCT Filed: Aug. 30, 2007
(86) PCT No.: PCT/AU07/01262

§ 371 Date: Feb. 8, 2011

Related U.S. Application Data
(60) Provisional application No. 60/902,355, filed on Feb. 21, 2007.

(51) Int. Cl. A61K 35/12; C12N 5/0783; G01N 33/50; A61P 7/00; A61P 7/06; A61P 37/02; A61P 3/10; A61P 37/06

(52) U.S. Cl. 424/93.71; 435/377; 435/7.24

(57) ABSTRACT

A method for generating a population of functional regulatory T cells (T_{REG}-cells), which are a subset of the T cell lineage having the ability to actively suppress immune activation and maintain peripheral immune tolerance, is described. The method comprises the steps of first culturing haemopoietic stem cells (HSC) and/or haemopoietic progenitor cells in the presence of a Notch ligand that supports T cell differentiation, and then isolating T cells having a T_{REG}-cell surface marker phenotype. A suitable source of HSC is cord blood (CB) and a suitable culture medium is OP9 cells engineered to express the Notch Ligand Delta-Like 1 (DL1) (OP9-DL1 cell line). Examples of T_{REG}-cell surface marker phenotypes are CD4+CD25+, CD45RO+, CD45RA+, CD127_{LOW}−, LAG-3 and/or CD39+.

### INCORPORATION BY REFERENCE

This patent application claims priority from:
U.S. Provisional Patent Application No. 60/902,355 entitled “Method for obtaining T_{REG}-cells” filed on 21 Feb. 2007.
   The entire content of this application is hereby incorporated by reference.

### FIELD OF THE INVENTION

The present invention relates to a method for obtaining T regulatory (T_{REG}) cells, in particular T_{REG}-cells having a CD4⁺CD25⁺ phenotype, from certain haemopoietic stem cells/progenitor cells present in cord blood.

### BACKGROUND OF THE INVENTION

Cord blood (CB) haemopoietic stem cells (HSC) are derived from the developing foetus and are found in the foetal side of the placental blood system. These cells have the capacity to form all blood cell types of the mature adult, and are therefore of enormous interest to medical researchers and developers of cell-based therapies. In particular, the use of cord blood HSC to produce T_{REG}-cells offers considerable potential for the development of cell-based immunosuppressive therapies for, inter alia, autoimmune diseases such as type I diabetes and rheumatoid arthritis (Sakaguchi, S. et al., 2006).

However, while the co-culture of cord blood HSC/haemopoietic progenitor cells on stromal feeders has facilitated the generation of a broad range of mature haemopoietic cells, considerable difficulty has been experienced in successfully generating cells of the lymphocyte lineage (Nakano, T. et al., 1994). More recently though, techniques to achieve differentiation of B lymphocytes from cord blood HSC, involving co-culture with a stromal feeder cell derived from an M-CSF deficient mouse (op/op) called OP9, has been developed (Carlyle, J. R. et al. 1997, Nakano, T. et al. 1994, and Nakano, T. et al. 1995). Further, following the identification of the critical role played by the so-called Notch signalling pathway in T lymphocyte development (Robey, E. et al., 1996, Washburn, T. et al., 1997, and Pui, J. C. et al., 1999), efficient in vitro generation of T cells can now be achieved. That is, through the co-culture of HSC or embryonic stem (ES) cells on OP9 stromal feeder cells expressing the Notch ligand, Delta-like 1 (DL1), it is now possible to efficiently produce T cells, in particular CD4⁺CD8⁺ T cells, in in vitro culture (de Pooter, R. F. et al., 2003, De Smedt, M. et al., 2004, and Schmitt, T. M. & J. C. Zuniga-Pflucker, 2002). The CD4⁺CD8⁺ T cells, otherwise known as CD4 CD8 double positive (DP) T cells, appear to be functionally similar to normal T cells and their development appears to correspond with “checkpoints” observed during in vivo thymopoiesis (La Motte-Mohs, R. N. et al., 2005, and Schmitt, T. M. et al., 2004, and Zakrzewski, J. L. et al., 2006).

Of particular interest to the present applicant however, are the T cells known as regulatory T cells (T_{REG}). Naturally occurring T_{REG}-cells (nT_{REG}), representing about 5-10% of circulating CD4⁺ T cells in mice and humans (Maloy, K. J. & F. Powrie, 2001, Sakaguchi, S. et al., 2001, and Gavin, M. & A. Rudensky, 2003), have the ability to actively suppress immune activation and maintain peripheral immune tolerance. Indeed, studies in several animal/pre-clinical models including type I diabetes and colitis, have shown that T_{REG}-cells are able to reduce disease status (Tang, Q. et al., 2004, and Uhlig, H. H. et al., 2006). Accordingly, the use of T_{REG}-cells in cell-based therapies of autoimmune diseases such as type I diabetes and rheumatoid arthritis and other inflammatory diseases, along with treatments for the prevention of transplant rejection, have been proposed. However, before such clinical uses can be developed, methods must be identified to allow for the in vitro generation of large numbers of T_{REG}-cells. To this end, methods have been proposed for the expansion of isolated natural T_{REG}-cells in vitro (Masteller, E. L. et al., 2006, and Bluestone, J. A. & Q. Tang, 2004). The present applicant however, hereinafter describes a novel method for generating large numbers of functional T_{REG}-cells through the in vitro differentiation of cord blood HSC/haemopoietic progenitor cells.

### SUMMARY OF THE INVENTION

In a first aspect, the present invention provides a method of obtaining a population of regulatory T cells (T_{REG}-cells), said method comprising the steps of;
(i) culturing haemopoietic stem cells (HSC) and/or haemopoietic progenitor cells in the presence of a Notch ligand that supports T cell differentiation, and thereafter
(ii) isolating T cells from the culture having a T_{REG}-cell surface marker phenotype.

T cells having a T_{REG}-cell surface marker phenotype isolated from the culture in accordance with the present invention will be enriched for T_{REG}-cells. Preferably, the T_{REG}-cell surface marker phenotype comprises a phenotype selected from the group consisting of: CD4⁺CD25⁺, CD45RO⁺, CD45RA⁺, CD127^{LOW/-}, LAG-3⁺, GPR83⁺ and/or CD39⁺. More preferably, the T_{REG}-cell surface marker phenotype is a CD4⁺CD25⁺ phenotype.

Most preferably, the isolated T cells having a T_{REG}-cell surface marker phenotype show a CD4⁺CD25⁺FOXP3⁺ phenotype.

Enrichment of T_{REG}-cells may be enhanced by culturing the cells in the presence of an enhancing agent such as interleukin-2 (IL-2). The population of cells derived from HSC cells may also be expanded by culturing the cells in the presence of an agent such as Fms-like tyrosine kinase 3 ligand (FLT3L) or interleukin-7 (IL-7).

In a second aspect, the present invention provides an isolated population of T cells expressing a T_{REG}-cell surface marker phenotype, obtained by the method of the first aspect.

In a third aspect, the present invention provides a T_{REG}-cell isolated from a population according to the second aspect.

The T_{REG}-cell of the third aspect is immunosuppressive, and in particular, can inhibit the proliferation of lymphocytes.

Thus, in a fourth aspect, the present invention provides a method of inhibiting the proliferation of a lymphocyte (particularly, a T cell), wherein said method comprises contacting the said lymphocyte with the T_{REG}-cell population of the second aspect or the T_{REG}-cell of the third aspect.

In a fifth aspect, the present invention provides a method of treating a subject for a disease for which immunosuppression may be desirable, wherein said method comprises administering (e.g. by infusion) to said subject the T_{REG}-cell population of the second aspect or the T_{REG}-cell of the third aspect, optionally in combination with a physiologically-acceptable carrier, excipient or diluent.

In a sixth aspect, the present invention provides a method of preventing transplant rejection, wherein said method comprises administering (e.g. by infusion) to a subject having received, or about to receive, a tissue transplant, the T_{REG}-cell population of the second aspect or the T_{REG}-cell of the third aspect, optionally in combination with a physiologically-acceptable carrier, excipient or diluent.

### BRIEF DESCRIPTION OF THE FIGURES

FIG. 1 provides graphical results showing cell differentiation of cord blood (CB) CD34⁺ HSC/progenitor cells towards CD4⁺CD25⁺ T_{REG}-cells. (a) CD4 and CD25 cell surface expression on CB HSC/progenitor cells cultured on OP9 cells (top panel), OP9 expressing DL1 (OP9-DL1) (middle panel) or OP9-DL1 with supplemental human interleukin-2 (hIL-2) (bottom panel) for 7, 14 or 21 days. For comparison, CD4 and CD25 profiles of natural T_{REG}-cells from freshly isolated CB mononuclear cells (MNC) are included. (b) Expansion of CB HSC-derived T_{REG}-cells (hpT_{REG}; closed symbols) and natural T_{REG}-cells (nT_{REG}; open symbols) cultured on OP9-DL1 for up to 21 days with or without supplemental hIL-2.

FIG. 2 provides graphical results showing that combinations of the cytokines Fms-like tyrosine kinase 3 ligand (FLT3L or FL), interleukin-7 (IL-7) and interleukin-2 (IL-2) promote expansion of CD34⁺ HSC/progenitor cells grown on OP9-DL1 cells. CD34⁺ HSC/progenitor cells were cultured on OP9-DL1 cells and cultures were supplemented with one of several combinations of the cytokines hFLT3L (FL), interleukin-7 (IL-7) and/or interleukin-2 (IL-2) for 7 days. Fold-expansion was then calculated based on the ratio of the total number of cells for the given cytokine combination (indicated below each bar) to the total number of cells in the absence of any of the cytokines (Supplementation of the culture by the relevant cytokine is indicated by a “+”, with absence indicated by a “−”).

FIG. 3 provides results from the assessment of the proportion of CD4+CD25+ T cells produced after 14 days culturing of CD34⁺ HSC/progenitor cells on OP9-DL1 supplemented with various cytokine combinations. CD34⁺ HSC/progenitor cells were cultured for 14 days on OP9-DL1 culture supplemented with FLT3L, IL-7 and either IL-2, IL-2 and transforming growth factor-β (TGF-β) or TGF-β. FACS analysis was performed to determine surface expression of CD4 and CD25. The left panel indicates the percentage of total cells which were CD4⁺CD25⁺ when supplemented with FLT3L, IL-7 and IL-2, the middle panel indicates the percentage of total cells which were CD4⁺CD25⁺ when supplemented with FLT3L, IL-7, IL-2 and TGF-β, and the right panel indicates the percentage of total cells which were CD4⁺CD25⁺ when supplemented with FLT3, IL-7 and TGF-β.

FIG. 4 provides results for the assessment of CD4⁺CD25⁺ T cells produced in accordance with the present invention, for the mRNA and protein expression of the Forkhead box P3 (FOXP3) transcription factor and immunosuppressive function. (a) Sorted populations of CD4⁺CD25⁺ T cells were assessed for FOXP3 mRNA expression by RT-PCR from fresh CB MNC (nT_{REG}), OP9-DL1 co-cultured natural T_{REG}-cells (OP9-DL1 nT_{REG}) and OP9-DL1 co-cultured CB CD34⁺ HSC/progenitor cells at day 14 (hpT_{REG}). A sorted population of CD4⁺CD25⁻ T cells was used as a negative control along with a no-template negative control. Total RNA samples were treated with DNaseI and reverse transcribed. Specific PCR products were then measured against the control gene Cyclophilin A. (b) OP9-DL1 co-cultured CB CD34⁺ cells were sorted for CD4⁺ and CD25⁺ expression at day 14. Sorted cells were then cultured on T_{REG} expander beads for 8 days. The histogram shows FOXP3 protein expression (shaded) in CD4⁺CD25⁺ cells compared to a matched isotype control antibody (white). (c) Immunosupressive capability of CB CD34⁺ HSC-derived CD4⁺CD25⁺ T_{REG}-cells was assessed by mixed lymphocyte reaction (MLR) assay. OP9-DL1 co-cultured CB CD34⁺ cells were sorted for CD4⁺ and CD25⁺ expression at day 14. Sorted cells were then cultured on T_{REG} expander beads for 8 days. Expanded CD4⁺ and CD25⁺ T_{REG}-cells and freshly isolated natural T_{REG} were cultured with antigen presenting cells (APCs), namely irradiated dendritic cells, anti CD3 (T cell activation molecule), and immuno-responder cells (CD4⁺CD25⁻; T effector) for 7 days at ratios of 1:1, 1:4 and 1:10, and the level of proliferation determined by tritiated thymidine incorporation.

FIG. 5 provides graphical indication that hpT_{REG}-cells show a mature phenotype based on surface expression analysis. CD34⁺ HSC/progenitor cells were cultured for 14 days on OP9-DL1 culture supplemented with FLT3L, IL-7 and IL-2 and analysed using FACS for surface expression of CD4 and MHC Class 2 molecules. Cells expressing CD4 also expressed MHC Class 2, indicating that such cells have a mature T cell phenotype.

FIG. 6 provides expression profiles of several transcription factors in hpT_{REG} cells. Sorted populations of CD4⁺CD25⁺ T cells were assessed for FOXP3, GATA3, TBET and RORgammaT mRNA expression by RT-PCR from OP9-DL1 co-cultured CB CD34⁺ HSC/progenitor cells at days 0, 7, 14 and 21 (hpT_{REG}). Total RNA samples were treated with DNaseI and reverse transcribed. Specific PCR products were then normalised against the control gene Cyclophilin A. The plot presents mean normalised expression of the transcription factors FOXP3 (circle, solid line), GATA3 (squares, short dashed line), TBET (triangle, long dash) and RORgammaT (“X”, dash-dot line).

### DETAILED DESCRIPTION OF THE INVENTION

Recent studies have revealed crucial roles of the Notch system in Th1, Th2 and T_{REG}-cell differentiation (Amsen, D. et al., 2004, Maekawa, Y. et al., 2003, Hoyne, G. F. et al., 2000, Vigouroux, S. et al., 2003, and Yvon, E. S. et al., 2003). For example, it has been independently demonstrated that Notch receptor activation by accessory cells can induce naive CD4⁺ T cells to develop as T_{REG}-cells (Hoyne, G. F. et al., 2000, and Yvon, E. S. et al., 2003). However, while these prior studies indicate that peripheral T cells are responsive to Notch signaling, considerable work is still required before the manner by which Notch signaling directs naive T cells toward the T_{REG}-cell fate is fully understood. Using the Notch ligand, DL1, the present applicant has found that it is possible to generate from certain haemopoietic stem cells/progenitor cells present in cord blood (CB), a population of functional T_{REG}-cells having a CD4⁺CD25⁺ phenotype that show similar characteristics to those of natural CB T_{REG}-cells. They have also found that it is possible to cause the significant enrichment of this T_{REG}-cell population by culturing in the presence of IL-2.

Thus, in a first aspect, the present invention provides a method of obtaining a population of regulatory T cells (T_{REG}-cells), said method comprising the steps of;
(i) culturing haemopoietic stem cells (HSC) and/or haemopoietic progenitor cells in the presence of a Notch ligand that supports T cell differentiation, and thereafter
(ii) isolating T cells from the culture having a T_{REG}-cell surface marker phenotype.

T cells having a T_{REG}-cell surface marker phenotype isolated from the culture in accordance with the present invention will be enriched for T_{REG}-cells. Preferably, the T_{REG}-cell surface marker phenotype comprises a phenotype selected from the group consisting of: CD4⁺CD25⁺, CD45RO⁺, CD45RA⁺, CD127^{LOW/-}, LAG-3⁺, GPR83⁺ and/or CD39⁺. More preferably, the T_{REG}-cell surface marker phenotype is a CD4⁺CD25⁺ phenotype.

Most preferably, the isolated T cells having a T_{REG}-cell surface marker phenotype show a CD4⁺CD25⁺FOXP3⁺ phenotype.

FOXP3, which is a nuclear protein believed to act as a transcriptional factor, is considered to provide a specific marker for T_{REG}-cells (Ramsdell, F. and S. F. Ziegler, 2003). However, since FOXP3 is an intracellular protein, it unfortunately cannot presently be used to separate T_{REG}-cells from a heterogeneous population (e.g. by using magnetic bead-based methods or cell sorting using a fluorescence-activated cell sorter (FACS)). It can, nevertheless, be used to assess a population of cells for the relative proportion of T_{REG}-cells present (i.e. quantification of the proportion of T_{REG}-cells in a population can be achieved by performing, for example, intracellular FOXP3 flow cytometry through permeabilising an aliquot of cells, thereafter staining with a labelled anti-FOXP3 antibody using any of the commercially available kits such as those available from eBioscience, Inc. (San Diego, Calif., United States of America) and BD Biosciences (San Jose, Calif., United States of America), and finally, undertaking FACS profiling to determine the percentage of cells expressing FOXP3 in the aliquot representative of the cell population).

The method of the first aspect of the present invention may involve culturing HSC and/or haemopoietic progenitor cells such as lymphoblasts and prolymphocytes that have been isolated, or partially purified, from cord blood (e.g. by using magnetic bead-based methods or cell sorting using a fluorescence-activated cell sorter (FACS)). Alternatively, haemopoietic progenitor cells may be isolated, or partially purified, from bone marrow, or otherwise produced following lineage-specific differentiation of embryonic stem (ES) cells.

However, preferably, the method involves culturing HSC, and particularly CD34⁺ HSC, that have been isolated from cord blood. CD34⁺ HSC may be isolated from cord blood using any of the methods well known to persons skilled in the art. One preferred method involves the isolation of CD34⁺ HSC from the fraction(s) of centrifuged cord blood which remain following removal of erythrocytes, by magnetic bead-based methods such as the magnetically activated cell sorting (MACS) protocol described in the CD34 MicroBead Kit from Miltenyi Biotec (Miltenyi Biotec GmbH, Cologne, Germany (2006)). The cord blood used to source the HSC will typically be human cord blood and may be derived from a specimen stored in a cord blood bank.

Preferably, the step of culturing the HSC and/or haemopoietic progenitor cells is conducted using a culture system comprising a suitable culture medium provided with the Notch ligand, DL1. However, since Notch receptors are able to bind Notch ligands “promiscuously”, other Notch ligands, for example Delta-like 4 (DL4) and jagged 2 (JAG2), that support T cell differentiation may also be suitable (Sambandam et. al. 2005, Bhandoola A, et. al. 2006, Bhandoola A, et. al. 2007). DL1 (or other Notch ligand) may be provided by simply adding suitable amounts of the purified protein to achieve a concentration which promotes T cell differentiation (e.g. 1-100 ng/ml). This concentration may, if desired, be maintained or adjusted as required throughout the duration of the culture.

Such a culture system may therefore be “cell free” (i.e. comprise no cells other than those intended to be cultured). However, conveniently, DL1 (or other Notch ligand) may be provided to the culture medium by the inclusion of suitable feeder cells.

Accordingly, the culture system may comprise a suitable culture medium that is provided with a population of a suitable feeder cell; such that the step of culturing the HSC and/or haemopoietic progenitor cells amounts to a co-culture of the HSC and/or haemopoietic progenitor cells and the feeder cells. Suitable feeder cells may include foetal liver stromal feeder cells such as AFT024 (Moore, K. A. et al., 1997), and bone marrow stromal feeder cells such as L87/4 and L88/5 (Thalmeier, K. et al., 1994), AC6.21 (Shih, C. C. et al., 1999) and FBMD-1 (Kusadasi, N. et al., 2000), which are well known to persons skilled in the art.

Preferably, the feeder cell is an OP9 bone marrow stromal feeder cell. This type of feeder cell does not, however, naturally express DL1 (or other Notch ligand such as DL4 and JAG2). Therefore, in a particularly preferred embodiment of the present invention, the culture medium comprises a population of an OP9 cell that has been transformed with, and stably expresses, an exogenous nucleic acid molecule encoding DL1 (designated OP9-DL1). In another particularly preferred embodiment of the present invention, the culture system comprises a population of a feeder cell derived from a human tissue source (e.g. a feeder cell derived from a human foreskin fibroblast cell or human thymus epithelial cell), particularly an autologous human tissue source.

Further, the culture system may comprise at least one enhancing agent to enhance the T cell differentiation or expansion that occurs during the step of culturing to thereby increase the relative amount of T_{REG}-cells within the isolated T cells having a T_{REG} surface marker phenotype. The enhancing agent may be selected from a range of different compounds. However, preferably, the enhancing agent is selected from suitable cytokines. More preferably, the enhancing agent is selected from IL-2, IL-7, interleukin-15 (IL-15), TGF-β, thymic stromal lymphopoietin (TSLP) and combinations thereof. Most preferably, the enhancing agent is selected from IL-2, IL-7, TSLP and combinations thereof. The enhancing agent will typically be provided in the culture medium at a concentration in the range of about 10 to 500 Units or 1-50 μg/ml. For IL-2, IL-7 and TSLP, the amount used will typically be in the range of about 10 to 500 Units.

Other growth/cell expansion factors such as Fms-like tyrosine kinase 3 ligand (FLT3L) may also be provided in the culture system.

In a particularly preferred embodiment of the invention, the culture system comprises FLT3L, IL-7 and IL-2. This combination of agents has been found to both expand the cell population and increase the percentage of T cells with a T_{REG}-cell surface marker phenotype present in the expanded population. These agents will typically be provided in the culture medium at concentrations in the range of 1-50 ng/ml for FLT3L and IL-7, and 100 U/ml for IL-2.

Moreover, the culture system may comprise dendritic cells (DCs), especially mature DCs, which have been reported to be capable of expanding CD4⁺CD25⁺ T cells in vitro (Yamazaki, S. et al., 2003).

The step of culturing the HSC and/or haemopoietic progenitor cells is preferably conducted using standard mammalian culture conditions for HSC cells. In one example, standard mammalian culture conditions comprise 2.5×10⁵ cells/ml in α-MEM media with 20% Fetal Calf Serum (FCS) at 37° C./5% CO₂. It will be understood by the person skilled in the art that variations made on the number of cells, media and percentage of FCS, temperature and CO₂ percentage may be made. Moreover, various alternatives to α-MEM medium may be used such as Dulbeco's Modified Eagles Medim (DMEM), Iscove's Modified Dulbecco's Media (Iscove's DMEM or IDMEM), and variants thereof which may include additional supplements such as L-glutamine. Serum-free or humanised alternatives may also be used. Under such conditions, and in the presence of a Notch ligand that supports T cell differentiation, T cells having a T_{REG}-cell surface marker phenotype (such as CD4⁺CD25⁺ T cells) may represent a transient population, and accordingly, the duration of the culturing step should be selected so as to coincide with the period during which T cells having a T_{REG}-cell surface marker phenotype are present.

Preferably, the duration of the culturing step is in the range of about 5 to 25 days, more preferably about 10 to 20 days, and most preferably, about 12 to 16 days. However, it has been found that as cell confluence increases, expression of the Notch ligand (e.g. DL1) by the feeder cells can be reduced, in which case, it may be desirable at one or more time points during the culturing step to reduce the level of cell confluence by any of the methods well known to persons skilled in the art (e.g. by “splitting” the feeder cell layers into halves and resuspending one or both of the halves in fresh culture medium).

The step of isolating T cells having a T_{REG}-cell surface marker phenotype (e.g. CD4⁺CD25⁺ T cells) from the culture may be conducted in accordance with any of the methods well known to persons skilled in the art, for example magnetic bead-based methods and FACS cell sorting techniques. For FACS cell sorting, the sorting or “gating” may preferably be conducted in a manner so as to isolate those cells present in the culture which show the appropriate T_{REG}-cell surface marker phenotype. For example, a high level of expression for both CD4⁺ and CD25⁺ (e.g. so-called CD25^{HIGH} T cells, where “high” represents the top 1-2% of expressors of CD25)). Further, such sorting may be based on the cells that express both CD4⁺ and CD25⁺ in the highest 20% of expressors, preferably, in the highest 10% of expressors, more preferably, in the highest 5% of expressors, and most preferably, in the highest 2% of expressors.

As mentioned above, T cells may be isolated according to those having a T_{REG}-cell surface marker phenotype. Examples of T_{REG}-cell surface marker phenotypes include a CD4⁺CD25⁺ phenotype, CD45RO⁺ phenotype (since it has been previously reported that CD4⁺CD25⁺ T cells that also express CD45RO possess “potent regulatory properties”; Jonuleit, H. et al., 2001; Seddiki, N. et al., 2006), a CD45RA⁺ phenotype (CD45RA⁺ is predominantly expressed on naïve T-cells, with expression switching from CD25RA+ to CD45RO+ phenotype on activation; Seddiki, N. et al., 2006), a CD127^{LOW} or CD127⁻ phenotype (Liu, W. et al., 2006), a LAG-3 (a CD4-related molecule that binds to MHC class II, and has been shown to be highly expressed in CD4⁺CD25⁺ T_{REG} cells; Bruder, D. et al., 2004, and Huang, C. T. et al., 2004) phenotype, a GPR83⁺ phenotype (Sugimoto, N et al., 2006) and/or a CD39⁺ phenotype (Borsellino, G. et al., 2007). Additionally or alternatively, the present invention may further comprise selection of cells based on combinations of these phenotypes. Moreover, sas mentioned above, the present invention may further comprise identifying and selecting T_{REG}-cells having a FOXP3+ phenotype.

In a second aspect, the present invention provides an isolated population of T cells expressing a T_{REG}-cell surface marker phenotype, enriched for T_{REG}-cells, obtained by the method of the first aspect.

As used herein, the term “enriched” means that the population of T cells expressing a T_{REG} surface marker phenotype comprises at least 25% T_{REG}-cells, more preferably at least 50% T_{REG}-cells, and most preferably, at least 75% T_{REG}-cells.

Preferably, the isolated population is obtained in accordance with the method of the first aspect.

In a third aspect, the present invention provides a T_{REG}-cell isolated from a population according to the second aspect.

Preferably, the T_{REG}-cell shows a CD4⁺CD25⁺FOXP3 phenotype. The T_{REG}-cell may also show a CD45RO⁺ phenotype, CD127^{LOW} or CD127⁻ phenotype, a LAG-3⁺ phenotype, a GPR83⁺ phenotype, and/or a CD39⁺ phenotype.

The T_{REG}-cell of the third aspect is immunosuppressive, and in particular, can inhibit the proliferation of lymphocytes.

Thus, in a fourth aspect, the present invention provides a method of inhibiting the proliferation of a lymphocyte (particularly, a T cell), wherein said method comprises contacting the said lymphocyte with the T_{REG}-cell population of the second aspect or the T_{REG}-cell of the third aspect.

In a fifth aspect, the present invention provides a method of treating a subject for a disease for which immunosuppression may be desirable, wherein said method comprises administering (e.g. by infusion) to said subject the T_{REG}-cell population of the second aspect or the T_{REG}-cell of the third aspect, optionally in combination with a physiologically-acceptable carrier, excipient or diluent.

The disease may be selected from autoimmune diseases such as type I diabetes, acquired haemolytic anaemia, pernicious anaemia, myasthenia gravis, glomerulonephritis, systemic lupus erythematosus (SLE), Sjögren's syndrome and rheumatoid arthritis and other inflammatory diseases.

In a sixth aspect, the present invention provides a method of preventing transplant rejection, wherein said method comprises administering (e.g. by infusion) to a subject having received, or about to receive, a tissue transplant, the T_{REG}-cell population of the second aspect or the T_{REG}-cell of the third aspect, optionally in combination with a physiologically-acceptable carrier, excipient or diluent.

The methods of the fourth, fifth and sixth aspects may further comprise the use of an immunosuppressive agent such as those well known to persons skilled in the art. Particularly suitable examples of such agents include cyclosporine, azathioprine, cyclophosphamide and prednisone.

Prior to use in the methods of the fifth and sixth aspects, the population of T cells expressing a T_{REG}-cell surface marker phenotype may, optionally, be treated so as to activate immunosuppressive function in the T_{REG}-cells. Such treatment may involve culturing the population in the presence of anti-CD3 antibodies. It is, however, considered that T_{REG}-cells produced in accordance with the present invention may show immunosuppressive function regardless of any specific activation treatment.

The present invention is hereinafter further described by way of the following, non-limiting examples and accompanying figures.

### EXAMPLES

### Example 1

### Methods and Materials

### Primary Cells and Cell Lines

Fresh primary human cord blood was obtained from volunteer donors. Mononuclear cells (MNC) were isolated by density gradient centrifugation over Lymphoprep™ solution (Axis-Shield, Oslo, Norway) and purified for CD34+ cells using magnetically activated cell sorting (MACS) with a Direct CD34 Progenitor Cell Isolation Kit and LS Separation Columns (Miltenyi Biotech, Auburn, Calif., United States of America).

An OP9 feeder cell line expressing DL1, designated OP9-DL1 (Schmitt, T. M. and J. C. Zuniga-Pflucker, 2002), was generated by infecting OP9 cells with a retroviral expression vector, pRUFpuro (Jenkins, B. J. et al., 1995), comprising a human DL1 gene, using standard methods.

### HSC/OP9-DL1 Co-Cultures

OP9-DL1 cells were prepared 16 hours prior to initiating co-cultures. The cells were seeded at 2×10⁴ cell/ml in 4 ml α-MEM media (Sigma-Aldrich Co., St Louis, Mo., United States of America) supplemented with 20% foetal calf serum (FCS) in 6 well plates (resulting in 8×10⁵ OP9-DL1 cells/well). Cord blood CD34⁺ cells or cord blood CD4⁺CD25⁺ cells were isolated by MACS enrichment and co-cultured at 2.5×10⁵ cells/ml on the pre-established OP9-DL1 stromal layer (80-90% confluent), in freshly prepared α-MEM media supplemented with 20% FCS, human recombinant (hr) FLT3L (10 ng/ml) and hr IL-7 (10 ng/ml) at 37° C./5% CO₂. Some co-cultures were also supplemented with hrIL-2 (100 U/ml). Haemopoietic cells were isolated using 40 μm nylon mesh filters and passaged every third day of culture onto pre-established OP9-DL1 stromal layers (prepared 16 hours earlier as described above) for up to 28 days.

### Cytofluorometry

For immunophenotyping of differentiated CB cells, anti-CD25 antibodies conjugated with phycoerythrin (PE), anti-CD8 antibodies conjugated to fluorescein isothiocyanate (FITC), anti-CD4 antibodies conjugated to phycoerythrin-Cy5 (PE-Cy5) and anti-MHC class 2 conjugated to phycoerythrin-Cy5 (PE-Cy5) were used (Becton, Dickinson and Company, San Jose, Calif., United States of America). Respective isotype controls were used. Samples were analysed on a flow cytometer (EPICS XL, Coulter, Miami, Fla., United States of America).

### Suppression Assay

HSC-derived CD4⁺CD25⁺ (hpT_{REG} where hp represents haemopoietic progenitor) and CD4⁺CD25⁻ cells were sorted after culture on OP9-DL1 for 14 days as described above. Sorted hpT_{REG} and natural T-reg (nT_{REG}) cells freshly isolated from CB by MACS cells were tested in an allo-MLR (based on the method described in Godfrey, W. R. et al., 2004) using unmatched 5×10⁴ CD25⁻ cells from a random donor peripheral blood mononuclear cell (PBMC) sample, and 3×10⁵ day 7 monocyte-derived dendritic cells (DCs) used as APCs cultured for 4-7 days. Proliferation was assessed by tritiated thymidine incorporation as previously described (Godfrey, W. R. et al., 2004).

### RT-PCR

Total RNA was prepared from haemopoietic cells using standard commercial reagents (TRIzol™, Life Technologies, Rockville, Md., United States of America). RNA was treated with DNase I (Ambion, Austin, Tex., United States of America), reverse transcribed using MMLV Reverse Transcriptase (QIAGEN, Valencia, Calif., United States of America) and quantitated by real time PCR using Taq polymerase (Amplitaq Gold, Applied Biosystems, Foster City, Calif., United States of America). Primers were designed to amplify PCR products with a TM of approx 65° C. PCR reactions were cycled at 60° C. for 10 minutes followed by 32 cycles of 95° C. for 30 seconds, 60° C. for 30 seconds and 72° C. for 30 seconds, with a final extension step of 90 seconds at 72° C. PCR products were run on ethidium agarose gels to ascertain specificity. Relative mRNA levels were quantitated against mRNA expression of Cyclophilin A.

### FOXP3 Protein Expression Analysis

Sorted CD4⁺CD25⁺ cells were analysed for FOXP3 protein expression by culturing on T_{REG} expander beads at a ratio of 2 cells per bead (Dynal®; Invitrogen Corporation, Carlsbad, Calif., United States of America) for 15 days in accordance with standard methods, and thereafter permeabilised and stained with a labelled anti-FOXP3 antibody using the FITC anti-human FOXP3 Fix/Perm Staining Set (eBioscience, Inc., San Diego, Calif., United States of America) in accordance with standard methods. Staining was compared to a matched isotype control antibody (Rat IgG₂ₐ) as provided in the FOXP3 Fix/Perm Staining Set.

### Results

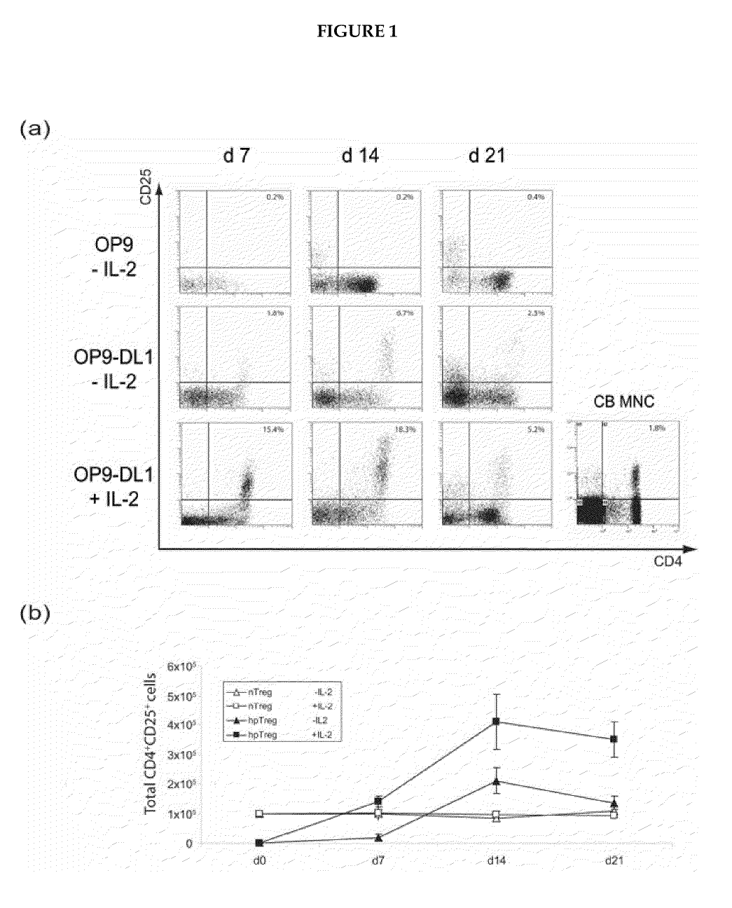

In a series of experiments (n=7), it was found that the co-culture of cord blood CD34⁺ HSC with OP9 cells expressing DL1 (OP9-DL1) in the presence of supplemental FLT3L (10 ng/ml) and IL-7 (10 ng/ml) predominantly supported the generation of CD4⁺CD8⁺ T cells (as previously described, La Motte-Mohs, R. N. et al., 2005), but also generated a previously unrecognised transient population of CD4⁺CD25⁺ cells peaking at day 14 of the culture (see FIG. 1a). This was surprising, because it is widely believed that CD4⁺CD25⁺ T cells are a late developing cell type (Ladi, E. et al., 2006, Wing, K. et al., 2006, Jiang, Q. et al., 2006).

The CD4⁺CD25⁺ T cells generated (i.e. hpT_{REG}-cells) displayed similar staining characteristics to populations of natural T_{REG}-cells (nT_{REG}) from freshly isolated CB MNC, were not observed in similar cultures of cord blood CD34⁺ HSC with OP9 cells which did not express DL1 (see FIG. 1a). When DL1 was present, the supplementation of the co-cultures with IL-2 (100 U/ml), resulted in a significant enhancement in the number of hpT_{REG}-cells generated (see FIG. 1a).

To confirm hpT_{REG}-cell differentiation from cord blood HSC/progenitor cells in the culture (rather than mere expansion of a contaminating CD4⁺CD25⁺ T cell population), purified natural CB T_{REG}-cells (showing a CD4⁺CD25⁺ FOXP3⁺ phenotype) were cultured on OP9-DL1 with and without IL-2. Whilst the numbers of cells were maintained for 14 days, this population did not significantly increase over the period of culture. In comparison, CB CD34⁺ HSC differentiation towards the CD4⁺CD25⁺ phenotype correlated with a significant cell expansion (see FIG. 1b), especially when IL-2 was present. No T_{REG}-cells were detectable in the CD34⁺ input population as determined by phenotypic markers. Total cell expansion in this system was up to 600 fold (n=5).

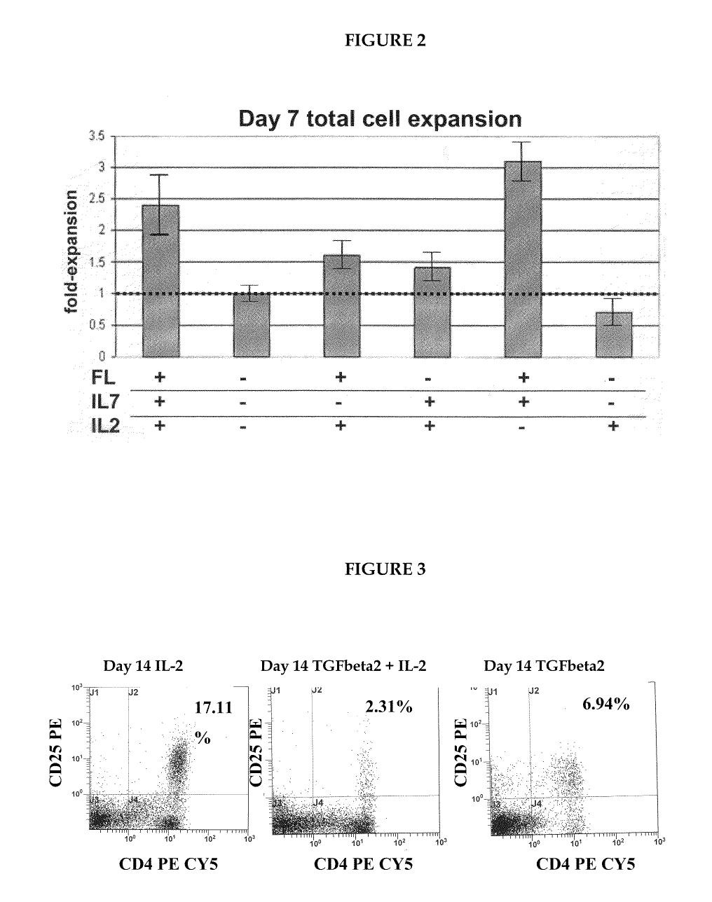

Expansion of CB CD34⁺ HSC in culture on OP9-DL1 cells was investigated using combinations of the cytokines Fms-like tyrosine kinase 3 ligand (FLT3L or FL), interleukin-7 (IL-7) and interleukin-2 (IL-2). CD34⁺ HSC/progenitor cells were cultured on OP9-DL1 cells and cultures were supplemented with one of several combinations of the cytokines FLT3L (FL), interleukin-7 (IL-7) and/or interleukin-2 (IL-2) for 7 days. Fold-expansion was then calculated based on the ratio of the total number of cells for the given cytokine combination (indicated below each bar) to the total number of cells in the absence of any of the cytokines, and the results are shown in FIG. 2. It can be seen that any combination including at least two of the three cytokines induced expansion of the cells after 7 days. In particular, FLT3L and IL-7 produced the largest expansion, followed by the combination of FLT3L, IL-7 and IL-2. When these results are compared to those of FIG. 1, it is apparent that whilst the expansion of cells by the combination of FLT3L, IL-7 and IL-2 is not as great as that obtained using FLT3L and IL-7, the relative fraction of CD4⁺CD25⁺ T_{REG} (hpT_{REG}) (to the total number of cells) is larger (i.e. is enhanced) when IL-2 is added (15.4% vs 1.8% at day 7 and 18.3% vs 6.7% at day 14).

Further experiments were also performed to investigate the effect of various cytokines on enhancing the production of hpT_{REG} cells. CD34⁺ HSC/progenitor cells were cultured for 14 days on OP9-DL1 culture supplemented with FLT3L, IL-7 and either IL-2, IL-2 and TGF-β, or TGF-β. FACS analysis was used to determine the percentage of CD4⁺CD25⁺ T_{REG} cells (hpT_{REG}s) and the results are presented in FIG. 3. The left panel indicates that 17% of cells were CD4⁺CD25⁺ when supplemented with FLT3L, IL-7 and IL-2, the middle panel indicates that 2.31% of cells were CD4⁺CD25⁺ when supplemented with FLT3L, IL-7, IL-2 and TGF-β, and the right panel indicates that 6.94% of the total cells were CD4⁺CD25⁺ when supplemented with FLT3L, IL-7 and TGF-β. These results can also be compared to FIG. 1, in which at day 14, 6.7% of cells were positive after growth in FLT3L and IL-7. Thus TGF-β had little effect on enhancing the production of hpT_{REG}-cells. In contrast, IL-2 produced much higher proportions of hpT_{REG}-cells in this system with the relative fraction being more than double that obtained compared to the case of no supplementation with IL-2.

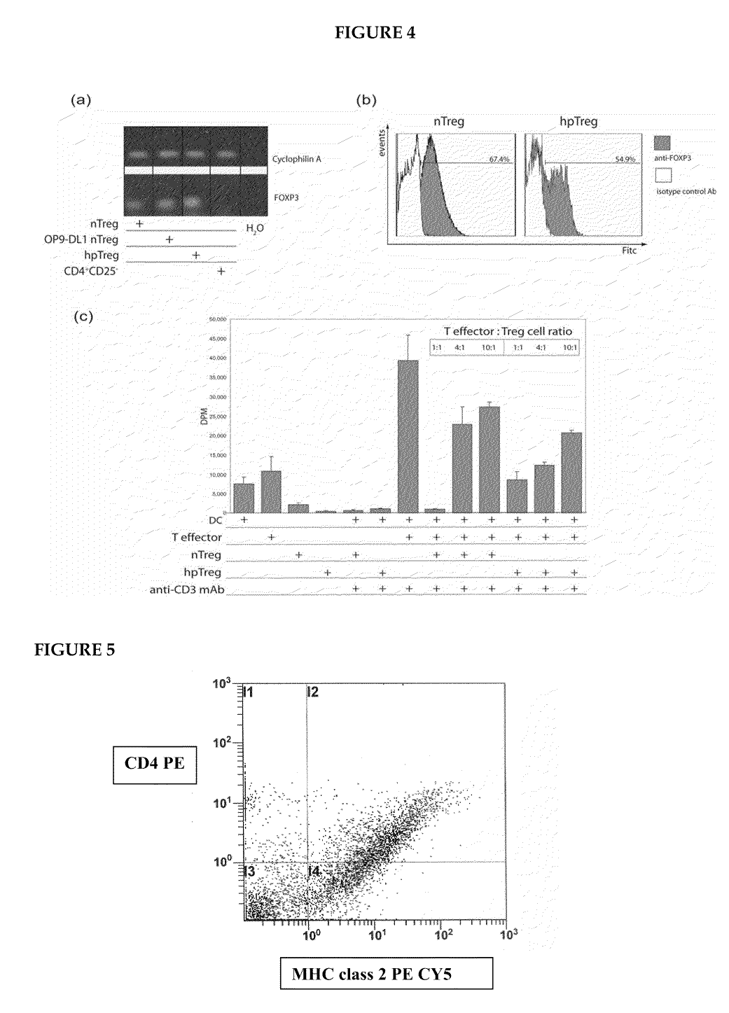

Whilst the CD4⁺CD25⁺ phenotype is known to enrich for T_{REG}-cells, CD25 is also expressed at low levels in a large proportion of circulating human T cells and is up-regulated after activation (Zola, H. et al., 1989), making it a non-exclusive marker for this T cell subset. However, since reconstitution experiments have demonstrated that the expression of FOXP3 during the thymic maturation of CD4⁺ T cells is essential for the production of T_{REG}-cells and correlates with T-reg immunosuppressive function (Ramsdell, F. and S. F. Ziegler, 2003, Fontenot, J. D. et al., 2003, Yagi, H. et al., 2004, Horis, S. et al., 2003, and Walker, M. R. et al., 2003), CD4⁺CD25⁺ T cells generated from OP9-DL1 co-cultured CB CD34⁺ HSC were assessed for the expression of FOXP3 using RT-PCR. That is, to assess FOXP3 mRNA expression in hpT_{REG}-cells, CB CD34+ HSCs were co-cultured on OP9-DL1 for 14 days, and RT-PCR performed on sorted CD4⁺CD25⁺ cells. As shown in FIG. 4a, hpT_{REG}-cells expressed a significant amount of FOXP3 mRNA that was consistent with the levels typically observed in either freshly isolated natural T_{REG}-cell populations, or from natural T_{REG}-cells after 14 days co-culture on OP9-DL1. To confirm FOXP3 protein expression, day 14 CD4⁺CD25⁺ T_{REG} cells were purified by flow cytometry and then cultured on human T-reg expander beads (Dynal™) for 8 days. Cells were then analysed by flow cytometry and, as shown in FIG. 4b, it was found that hpT_{REG}-cells express significant levels of FOXP3 protein, comparable with expression in naturally derived T_{REG}-cells (55%+ve vs 67%+ve respectively).

Importantly, hpT_{REG}-cells actively suppressed cell proliferation when cultured with antigen presenting cells (APCs) and immune responders (i.e. cord blood CD4⁺CD25⁻ T effector cells) compared to CD4⁺CD25⁻ T effector cells in a suppression assay utilising a mixed lymphocyte reaction (MLR) (see FIG. 4c). In this assay, it was observed that the hpT_{REG}-cells were anergic (see column 4), resistant to activation by anti-CD3 antibodies (see column 6) and were potent suppressors of proliferating CD4⁺CD25⁻ T effector cells (see columns 11-13). In comparison with freshly isolated nT_{REG}-cells (see columns 8-10), the hpT_{REG}-cells were at least as potent suppressors of proliferation as nT_{REG}-cells (hpT_{REG} 52.8% suppression vs nT_{REG} 30.2% suppression at 1:10 T_{REG}:responder). These results strongly indicate that the cells produced in this system are akin to nT_{REG}-cells, and not an activation induced transient T_{REG}-cell population (Allan, S. E. et al., 2007).

To further investigate that hpT_{REG}-cells were mature equivalents to natural T_{REG}-cells, CD34⁺ HSC/progenitor cells were cultured for 14 days on OP9-DL1 culture supplemented with FLT3L, IL-7 and IL-2 and analysed using FACS for surface expression of CD4 and MHC Class 2 molecules. FIG. 5 presents the FACS plot indicating that cells expressing CD4 also expressed MHC Class 2 (at typically brighter levels), indicating that the hpT_{REG}-cells have a mature T cell phenotype.

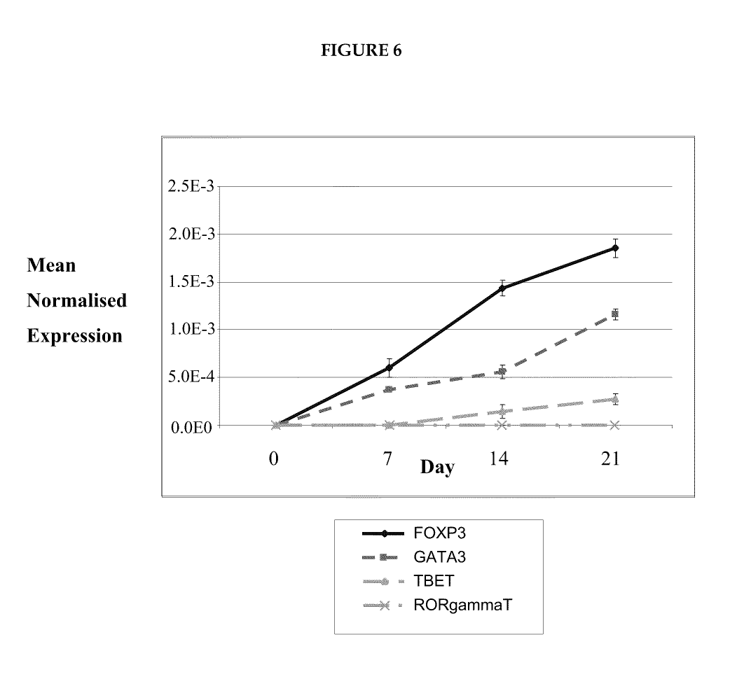

Expression profiles of several transcription factors was also performed in hpT_{REG}-cells. Sorted populations of CD4⁺CD25⁺ T cells were assessed for FOXP3, GATA3, TBET and RORgammaT mRNA expression by RT-PCR from OP9-DL1 co-cultured CB CD34⁺ HSC/progenitor cells at days 0, 7, 14 and 21 (hpT_{REG}). Total RNA samples were treated with DNaseI and reverse transcribed. Specific PCR products were then normalised against the control gene Cyclophilin A. Results are presented in FIG. 6 and the plot presents mean normalised expression of the transcription factors FOXP3 (circle, solid line), GATA3 (squares, short dashed line), TBET (triangle, long dash) and RORgammaT (“X”, dash-dot line). It is apparent that expression of FOXP3 is greater than expression of these other factors, with there being no detectable expression of RORgammaT. Further, to rule out failure of primers in the case of RORgammaT, they were tested in an independent experiment (data not shown) and detectable levels of RORgammaT were found indicating that the primers were suitable.

Thus, the results of FIGS. 4 to 6 indicate that the CD4⁺CD25⁺ T_{REG} cells (hpT_{REG}) are mature equivalents to natural T_{REG} cells, showing high expression of FOXP3 and suppression capability.

Surface profiling for a range of markers was also performed on CD4⁺CD25⁺ FOXP3+ cells. Cells at day 14 were selected using FACS and the percentage of these cells expressing CD127, MHCII, CD39, CD45RO and CTLA4 assessed. The results are presented in Table 1. The CD4⁺CD25⁺FOXP3⁺ cells showed low levels of CD127, and high levels of CD39 and CD45RO consistent with previously observed T_{REG}-cell surface marker phenotypes (Jonuleit, H. et al., 2001; Seddiki, N. et al., 2006, Liu, W. et al., 2006, Borsellino, G. et al., 2007).

TABLE 1
Surface expression of several markers on hpT_{REG} cells

| | % of |
|---|---|
| | CD4⁺CD25⁺FOXP3⁺ cells |
| | positive for the given |
| Surface Marker | surface marker |
| CD127 | 4.6% |
| MHCII | 45% |
| CD39 | 94.3% |
| CD45RO | 96.2% |
| CTLA4 | 40% |

### Discussion

T_{REG}-cells from cord blood are potent suppressors of immune responses to a wide variety of antigens, and are capable of reversing the destructive consequence of autoimmune diseases such as type I diabetes and rheumatoid arthritis. Recently, the role of Notch ligands in lymphoid differentiation has been confirmed using in vitro assays on OP9 stromal cells. Delta-like 1 (DL1) signalling has been shown to drive CD4⁺CD8⁺ T cell differentiation of embryonic stem cells, adult haemopoietic progenitors and cord blood haemopoietic progenitor cells. This example shows the development of a transient population of CD4⁺CD25⁺FOXP3⁺ T cells (although other suitable surface markers for T_{REG} cells could have been utilised), having similar characteristics to those of natural CB T_{REG}-cells, that emerges in co-cultures of CB HSC/progenitor cells and OP9 cells expressing DL1. Further, it has been shown that the development of these cells can be significantly enhanced by IL-2 especially when combined with FLT3L and IL-7. The culture system therefore represents an important advance in the production of large numbers of T_{REG}-cells to enable the development of cell-based therapies for the treatment of autoimmune diseases and prevention of transplant rejection.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Allan S. E. et al., Activation-induced FOXP3 in human Teffector cells does not suppress proliferation or cytokine production. Int Immunol., 19:345-354 (2007).
2. Amsen, D. et al., Instruction of distinct CD4 T helper cell fates by different notch ligands on antigen-presenting cells. Cell, 117(4):515-526 (2004).
3. Bhandoola A, et al., Commitment and developmental potential of extrathymic and intrathymic T cell precursors: plenty to choose from. Immunity. 2007; 26:678-689.
4. Bhandoola A. and A. Sambandam, From stem cell to T cell: one route or many? Nat Rev Immunol. 2006; 6:117-126.
5. Bluestone, J. A. and Q. Tang, Therapeutic vaccination using CD4+CD25+ antigen-specific regulatory T cells. Proc Natl Acad Sci USA, 101 Suppl 2:14622-14626 (2004).
6. Borsellino, G. et al., Expression of ectonucleotidase CD39 by Foxp3⁺ Treg cells: hydrolysis of extracellular ATP and immune suppression. Blood, 110(4):1225-1232 (2007).
7. Bruder, D. et al., Neuropilin-1: a surface marker of regulatory T cells. Eur J Immunol, 34:623-630 (2004).
8. Carlyle, J. R. et al., Identification of a novel developmental stage marking lineage commitment of progenitor thymocytes. J Exp Med. 186(2):173-182 (1997).
9. de Pooter, R. F. et al., In vitro generation of T lymphocytes from embryonic stem cell-derived prehematopoietic progenitors. Blood, 102(5):1649-1653 (2003).
10. De Smedt, M. et al., Human bone marrow CD34+ progenitor cells mature to T cells on OP9-DL1 stromal cell line without thymus microenvironment. Blood Cells Mol Dis, 33(3):227-232 (2004).
11. Fontenot, J. D. et al., Foxp3 programs the development and function of CD4+CD25+ regulatory T cells. Nat Immunol, 4(4):330-336 (2004).
12. Gavin, M. and A. Rudensky, Control of immune homeostasis by naturally arising regulatory CD4+ T cells. Curr Opin Immunol, 15(6):690-696 (2003).
13. Godfrey, W. R. et al., In vitro-expanded human CD4(+)CD25(+) T-regulatory cells can markedly inhibit allogeneic dendritic cell-stimulated MLR cultures. Blood, 104(2):453-461 (2004).
14. Hori, S. et al., Control of regulatory T cell development by the transcription factor Foxp3. Science, 299(5609):1057-1061 (2003).
15. Hoyne, G. F. et al., Serratel-induced notch signalling regulates the decision between immunity and tolerance made by peripheral CD4(+) T cells. Int Immunol, 12(2):177-185 (2000).
16. Huang, C. T. et al., Role of LAG-3 in regulatory T cells. Immunity, 21:503-513 (2004).
17. Jenkins, B. J. et al., Activating point mutations in the common β subunit of the human GM-CSF, IL-3 and IL-5 receptors suggest the involvement of β subunit dimerization and cell type-specific molecules in signalling. EMBO J, 14:4276-4287 (1995).
18. Jiang, Q. et al., Delayed functional maturation of natural regulatory T cells in the medulla of postnatal thymus: role of TSLP, BMC Immunol, 7:6 (2006).
19. Jonuleit, H. et al., Identification and functional characterization of human CD4(+)CD25(+) T cells with regulatory properties isolated from peripheral blood. J Exp Med, 193(11):1285-1294 (2001).
20. Kögler, G. et al. The effect of different thawing methods, growth factor combinations and media on ex vivo expansion of umbilical cord blood primitive and committed progenitors. Bone Marrow Transplant, 21:233-241 (1998).
21. Kusadasi, N. et al., Successful short-term ex vivo expansion of NOD/SCID repopulating ability and CAFC week 6 from umbilical cord blood. Leukemia, 14(11):1944-1953 (2000).
22. Ladi E. et al., Thymic microenvironments for T cell differentiation and selection, Nature Immunology, 7:338-343 (2006).
23. La Motte-Mohs, R. N. et al., Induction of T-cell development from human cord blood hematopoietic stem cells by Delta-like 1 in vitro. Blood, 105(4):1431-1439 (2005).
24. Liu, W. et al., CD127 expression inversely correlates with FOXP3 and suppressive function of human CD4⁺ T reg cells. JEM, 203(7):1701-1711 (2006).
25. Maekawa, Y. et al., Delta1-Notch3 interactions bias the functional differentiation of activated CD4+ T cells. Immunity, 19(4): 549-559 (2003).
26. Maloy, K. J. and F. Powrie, Regulatory T cells in the control of immune pathology. Nat Immunol, 2(9):816-822 (2001).
27. Masteller, E. L. et al., Antigen-specific regulatory T cells—ex vivo expansion and therapeutic potential. Semin Immunol, 18 (2) 103-110 (2006).
28. Miltenyi Biotec GmbH, CD34 Microbead Kit, DS130-046-702-3, 2007, http://www.miltenyibiotec.com/en/PG_{—}625_{—}2_CD34_MicroBead_Kit.asp x.
29. Moore, K. A. et al., In vitro maintenance of highly purified, transplantable hematopoietic stem cells. Blood, 89(12):4337-4347 (1997).
30. Nakano, T. et al., Generation of lymphohematopoietic cells from embryonic stem cells in culture. Science, 265(5175):1098-1101 (1994).
31. Nakano, T. et al., Lymphohematopoietic development from embryonic stem cells in vitro. Semin Immunol, 7(3):197-203 (1995).
32. Pui, J. C. et al., Notch1 expression in early lymphopoiesis influences B versus T lineage determination. Immunity, 11(3):299-308 (1999).
33. Ramsdell, F. & S. F. Ziegler, Transcription factors in autoimmunity. Curr Opin Immunol, 15(6):718-724 (2003).
34. Robey, E. et al., An activated form of Notch influences the choice between CD4 and CD8 T cell lineages. Cell, 87(3):483-492 (1996).
35. Sakaguchi S. et al., Foxp3+CD25+CD4+ natural regulatory T cells in dominant self-tolerance and autoimmune disease. Immunol Rev, 212:8-27 (2006).
36. Sakaguchi, S. et al., Immunologic tolerance maintained by CD25+CD4+ regulatory T cells: their common role in controlling autoimmunity, tumor immunity, and transplantation tolerance. Immunol Rev, 182:18-32 (2001).
37. Sambandam A. et al., Notch signaling controls the generation and differentiation of early T lineage progenitors. Nat. Immunol. 2005; 6:663-670.
38. Schmitt, T. M. and J. C. Zuniga-Pflucker, Induction of T cell development from hematopoietic progenitor cells by delta-like-1 in vitro. Immunity, 17(6):749-756 (2002).
39. Schmitt, T. M. et al., Induction of T cell development and establishment of T cell competence from embryonic stem cells differentiated in vitro. Nat Immunol, 5(4):410-417 (2004).
40. Seddiki, N. et al., Persistence of naive CD45RA⁺ regulatory T cells in adult life. Blood, 107(7):2830-2838 (2006).
41. Shih, C. C. et al., Long-term ex vivo maintenance and expansion of transplantable human hematopoietic stem cells. Blood, 94(5):1623-1636 (1999).
42. Sugimoto, N. et al., Foxp3-dependent and -independent molecules specific for CD25⁺CD4⁺ natural regulatory T cells revealed by DNA microarray analysis, International Immunology, 18(8):1197-1209 (2006).
43. Tang Q. et al. In vitro-expanded antigen-specific regulatory Tcells suppress autoimmune diabetes. J Exp Med., 199:1455-1465 (2004).
44. Thalmeier, K. et al., Establishment of two permanent human bone marrow stromal cell lines with long-term post irradiation feeder capacity. Blood, 83(7):1799-1807 (1994).
45. Uhlig H. H. et al., Characterization of Foxp3+CD4+CD25+ and IL-10-secreting CD4+CD25+ T cells during cure of colitis. J. Immunol., 177:5852-5860 (2006).
46. Vigouroux, S. et al., Induction of antigen-specific regulatory T cells following overexpression of a Notch ligand by human B lymphocytes. J Virol, 77(20):10872-10880 (2003).
47. Walker, M. R. et al., Induction of FoxP3 and acquisition of T regulatory activity by stimulated human CD4+CD25− T cells. J Clin Invest, 112(9):1437-1443 (2003).
48. Washburn, T. et al., Notch activity influences the alphabeta versus gammadelta T cell lineage decision. Cell, 88(6):833-843 (1997).
49. Wing, K. et al., Emerging possibilities in the development and function of regulatory T cells. Int Immunol, 18(7):991-1000 (2006).
50. Yagi, H. et al., Crucial role of FOXP3 in the development and function of human CD25+CD4+ regulatory T cells. Int Immunol, 16 (141643-1656 (2004).
51. Yamazaki, S. et al., Direct expansion of functional CD25⁺CD4⁺ regulatory T cells by antigen-processing dendritic cells. J Exp Med, 198(2):235-247 (2003).
52. Yvon, E. S. et al., Overexpression of the Notch ligand, Jagged-1, induces alloantigen-specific human regulatory T cells. Blood, 102(10):3815-3821 (2003).
53. Zakrzewski, J. L. et al., Adoptive transfer of T-cell precursors enhances T-cell reconstitution after allogeneic hematopoietic stem cell transplantation. Nat Med, 12(9):1039-1047 (2006).
54. Zola, H. et al., Circulating human T and B lymphocytes express the p55 interleukin-2 receptor molecule (TAC, CD25). Immunol Cell Biol, 67(4):233-237 (1989).

1. A method of obtaining a population of regulatory T cells (T_{REG}-cells), said method comprising the steps of
(i) culturing haemopoietic stem cells (HSC) and/or haemopoietic progenitor cells in the presence of a Notch ligand that supports T cell differentiation, and thereafter
(ii) isolating T cells from the culture having a T_{REG}-cell surface marker phenotype.

2. The method of claim 1, wherein the T_{REG}-cell surface marker phenotype includes one or more surface marker phenotypes selected from the group consisting of: CD4⁺CD25⁺, CD45RO⁺, CD45RA⁺, CD127^{LOW/-}, LAG-3⁺, GPR83⁺ and/or CD39⁺.

3. The method of claim 2, wherein the T_{REG}-cell surface marker phenotype is a CD4⁺CD25⁺ phenotype.

4. The method of claim 1, wherein said HSC and/or haemopoietic progenitor cells have been isolated, or partially purified, from cord blood.

5. The method of claim 1, wherein said culturing step comprises culturing isolated CD34⁺ HSC.

6. The method of claim 1, wherein said culturing step comprises culturing the HSC and/or haemopoietic progenitor cells in a cell-free culture system comprising a suitable culture medium provided with an amount of Delta-like 1 (DL1), or another Notch ligand, that supports T cell differentiation.

7. The method of claim 1, wherein said culturing step comprises culturing the HSC and/or haemopoietic progenitor cells in a culture system comprising a suitable culture medium and feeder cells providing an amount of Delta-like 1 (DL1), or another Notch ligand, that supports T cell differentiation.

8. The method of claim 7, wherein the feeder cells are derived from a human tissue source.

9. The method of claim 6, wherein the culture system further comprises at least one enhancing agent to enhance T cell differentiation during the said culturing step to thereby increase the relative amount of T_{REG}-cells within the culture system.

10. The method of claim 9, wherein the enhancing agent is selected from interleukin 2 (IL-2), interleukin 7 (IL-7), interleukin 15 (IL-15), transforming growth factor-β (TGF-β), thymic stromal lymphopoietin (TSLP) and combinations thereof.

11. The method of claim 10, wherein the enhancing agent is selected from IL-2, IL-7, TSLP, and combinations thereof.

12. The method of claim 11, wherein the enhancing agent is IL-2.

13. The method of claim 6, wherein the culture system further comprises Fms-like tyrosine kinase 3 ligand (FLT3L).

14. The method of claim 6, wherein the culture system further comprises Fms-like tyrosine kinase 3 ligand (FLT3L), interleukin 2 (IL-2) and interleukin 7 (IL-7).

15. The method of claim 1, wherein said culturing step is conducted using standard mammalian culture conditions for supporting HSC cells, for a duration in the range of about 10 to 20 days.

16. The method of claim 1, wherein said culturing step is conducted using standard mammalian culture conditions for supporting HSC cells, for a duration in the range of about 12 to 16 days.

17. The method of claim 15, wherein said standard mammalian cultures for supporting HSC cells are 2.5×10⁵ cells/ml in α-MEM media with 20% FCS at 37° C./5% CO₂.

18. The method of claim 1, wherein the method further comprises identifying and selecting T_{REG}-cells having a FOXP3⁺ phenotype.

19. The method of claim 1, wherein the method further comprises identifying and selecting T_{REG}-cells having a CD4⁺CD25⁺ phenotype, CD45RO⁺ phenotype, CD45RA⁺ phenotype, a CD127^{LOW/-} phenotype, a LAG-3 phenotype, a GPR83⁺ phenotype and/or a CD39⁺ phenotype.

20. An isolated population of T cells expressing a T_{REG}-cell surface marker phenotype, enriched for T_{REG}-cells, obtained by the method of claim 1.

21. A T_{REG}-cell isolated from the population of claim 20.

22. The T_{REG}-cell of claim 21, having a CD4⁺CD25⁺ phenotype.

23. The T_{REG}-cell of claim 22, having a CD4⁺CD25⁺FOXP3⁺ phenotype.

24. The T_{REG}-cell of claim 21, having a CD45RO⁺ phenotype, CD45RA⁺ phenotype, a CD127^{LOW/-} phenotype, a LAG-3 phenotype, a GPR83⁺ phenotype, and/or a CD39⁺ phenotype.

25. A method of inhibiting the proliferation of a lymphocyte, wherein said method comprises contacting the said lymphocyte with the T_{REG}-cell population of claim 20 or a T_{REG}-cell isolated from said population and having a phenotype selected from a CD4⁺CD25⁺ phenotype, a CD4⁺CD25⁺FOXP3⁺ phenotype, a CD45RO⁺ phenotype, CD45RA⁺ phenotype, a CD127^{LOW/-} phenotype, a LAG-3 phenotype, a GPR83⁺ phenotype, and/or a CD39⁺ phenotype.

26. A method of treating a subject for a disease for which immunosuppression may be desirable, wherein said method comprises administering to said subject the T_{REG}-cell population of claim 20 or a T_{REG}-cell isolated from said population and having a phenotype selected from a CD4⁺CD25⁺ phenotype, a CD4⁺CD25⁺FOXP3⁺ phenotype, a CD45RO⁺ phenotype, CD45RA⁺ phenotype, a CD127^{LOW/-} phenotype, a LAG-3 phenotype, a GPR83⁺ phenotype, and/or a CD39⁺ phenotype, optionally in combination with a physiologically-acceptable carrier, excipient or diluent.

27. The method of claim 26, wherein the disease is selected from type I diabetes, acquired haemolytic anaemia, pernicious anaemia, myasthenia gravis, glomerulonephritis, systemic lupus erythematosus (SLE), Sjögren's syndrome, rheumatoid arthritis and other inflammatory diseases.

28. A method of preventing transplant rejection, wherein said method comprises administering to a subject having received, or about to receive, a tissue transplant, the T_{REG}-cell population of claim 20 or a T_{REG}-cell isolated from said population and having a phenotype selected from a CD4⁺CD25⁺ phenotype, a CD4⁺CD25⁺FOXP3⁺ phenotype, a CD45RO⁺ phenotype, CD45RA⁺ phenotype, a CD127^{LOW/-} phenotype, a LAG-3 phenotype, a GPR83⁺ phenotype, and/or a CD39⁺ phenotype, optionally in combination with a physiologically-acceptable carrier, excipient or diluent.

29. The method of claim 25, further comprising administering to said subject, an immunosuppressive agent.
